(19) United States
(12) Patent Application Publication
(10) Pub. No.: US 2013/0056010 A1
(43) Pub. Date: Mar. 7, 2013

(54) AUTONOMOUS POSITIVE AIRWAY PRESSURE SYSTEM

(71) Applicants: Elijah Charles Walker, Silver Spring, MD (US); Anthony P. Kimani Mwangi, Atlanta, GA (US)

(72) Inventors: Elijah Charles Walker, Silver Spring, MD (US); Anthony P. Kimani Mwangi, Atlanta, GA (US)

(21) Appl. No.: 13/634,385

(22) PCT Filed: Mar. 10, 2011
(86) PCT No.: PCT/US11/27890

§ 371 Date: Nov. 21, 2012

Related U.S. Application Data
(60) Provisional application No. 61/313,308, filed on Mar. 12, 2010.

(51) Int. Cl. A61F 5/56; A61M 16/20; A61M 16/16; A61M 16/14; A61M 16/00; A61M 15/08

(52) U.S. Cl. 128/848; 128/205.22; 128/203.12; 128/204.17; 128/202.16

(57) ABSTRACT

A self-contained Autonomous Positive Air Pressure (APAP) device that treats sleep apnea by providing pressurized air intermittently or on demand. The self-contained device is inconspicuously designed as eyewear and does not require the user to be tethered to an air hose or power cord. The device also does not require the user to wear a face mask sealed to the user's face. Instead, the present invention comprises a system that is battery powered including pumps to pressurize a chamber or chambers that provides breathable air through electronically controlled pressure regulated valves that prevents upper airway obstruction.

### CROSS-REFERENCE TO RELATED APPLICATIONS

This application claims priority from provisional application Ser. No. 61/313,308 filed Mar. 12, 2010, which is incorporated herein by reference.

### FIELD OF THE INVENTION

The present application pertains to positive airway pressure for treating sleep-related breathing disorders. More particularly, this application pertains to a self-contained, air hose-free device to enhance breathing during sleep by preventing and alleviating upper airway obstruction and restriction resulting from sleep-related breathing disorders such as snoring, obstructive sleep apnea, obstructive sleep hypopnea, or upper airway resistance.

### BACKGROUND OF THE INVENTION

Sleep apnea (apnea meaning a cessation of airflow) is a relatively common and potentially life-threatening sleep disorder that impacts millions of people in the United States and around the world.

Obstructive sleep apnea (OSA), obstructive sleep hypopnea, and upper airway resistance are characterized by upper airway abnormalities that result in airway collapse and complete or partial obstruction of airflow into the lungs. Upper airway (i.e. upper respiratory tract, or airway) abnormalities include: a smaller (than normal) airway cross-sectional area that subjects the pharynx to collapse; an enlarged tongue that can obstruct the airway by moving posteriorly (backward) into airway space during sleep; a retruding jaw that can increase tissue pressure surrounding the airway and subject it to collapse; an enlarged soft palate that can impinge on airway space when breathing; or compromised pharyngeal dilator muscles that fail to keep the airway open when inhaling, causing momentary obstruction of airflow Powered Apparatus Approaches.

Various apparatus-based approaches (e.g. non-surgical, and non-pharmacological) have been developed to treat snoring and/or sleep apnea that in general can be divided into: (a) apparatus that require a power source (Powered) and (b) apparatus that do not require a power source (non-powered). Apparatus that require power sources (usually involving forced ventilation) include medical devices, such as Continuous Positive Airway Pressure (CPAP) devices, and negative pressure apparatus. Those who dislike CPAP give many reasons including: mask discomfort, difficulty adapting to the pressure, dislike being tethered to a machine, nasal irritation, sore throat, and allergies.

Non-powered apparatus (typically oral appliances) offer additional solutions for snoring or sleep apnea. Oral appliances can generally be separated into two types: Mandibular Repositioning Appliances such as disclosed in U.S. Pat. No. 6,729,335, to Halstrom; and Tongue Retainer appliances. Mandibular Repositioning Appliances (MRAs, sometimes known as mandibular advancement appliances) purport to reposition the mandible anteriorly to further open the airway to prevent its obstruction.

Powered apparatus, typically CPAP devices, suffer from a number of shortcomings, including low user compliance caused by a number of reasons, such as: lack of easy portability, device appearance, excessive air pressure, tight face masks or devices that require the user to be tethered to an air hose that is connected to the user's face mask. A tethered device can limit the user's range of motion and potentially disturb sleep.

### SUMMARY OF THE INVENTION

In accordance with the present invention, there is provided a self-contained device Autonomous Positive Air Pressure (APAP) System that treats sleep apnea by providing positive pressurized gas in an intermittent, timed or on-demand basis. As used herein, “gas” includes any breathable gas, including ambient air and air that is enriched with one or more beneficial components, such as water, pharmaceutical components, medicants, or the like.

A self-contained device in embodiments of the invention does not require the user to be tethered to an air hose or power cord or other means. Such device also does not require the user to wear a facemask over the nose that can be dislodged during sleep.

Instead, the present invention comprises in embodiments an inconspicuous system contained in a single unit that is battery powered. An exemplary embodiment includes eyewear worn by the user comprising a tank(s) or chamber(s) that contain gas and can be pressurized with pumps and provide breathable air to a user through electronically controlled pressure regulated valves. During device usage the chamber can remain pressurized by the pumps and serves as an air supply tank to provide breathable air to the user that minimizes the use of the pumps thus helping to conserve battery power. The pressure-regulated valves are connected to flexible nasal tubes that supply air to the user's nasal or oral cavity. The pressure and flow of gas delivered to the user (quantity and volume/time as well as change of volume/time) is electronically and pneumatically controlled and can vary to include a variety of waveforms. The gas chamber is ergonomically and aesthetically integrated into the structure of the device to minimize space requirements and improve device appearance. The eyewear also comprises comfortable material(s) to interface and conform to the users body form and can evenly distribute pressure from the eyewear across the users body (e.g. face), provide softness to improve the feel of the device, and potentially absorb secretions (such as sweat) from the user's body.

In embodiments, an apparatus of the invention may be placed in a variety of locations including, without limitation, to the face, such as in the form of eyeglasses or goggles comprising dual lenses configured to serve as the gas chamber, or as headgear where the chamber is contoured to fit on the user's head and includes all components required to operate the APAP, or the device may be configured to be placed on other locations such as the arm, torso, back, or leg. In alternative embodiments one or more of the device components, including device power source, may be remotely located in other locations. The eyewear or headgear can also comprise sensors such as accelerometers to detect body position or rate of movement, microphone to detect sound, EEG sensors to detect sleep stage, pulse oximeter to detect oxygen saturation, nasal or oral pressure transducer and/or thermistors to detect nasal pressure or oral pressure or temperature or airflow in order to detect changes in user airflow such as inspiration, expiration, or apnea or hypopnea events. In some embodiments, when changes in user airflow is detected such as inspiration, or apnea or hypopnea events, an electronic circuit may trigger the device to release pressurized air into the nasal or oral cavity thus assisting breathing or mitigating or stopping the apnea or hypopnea event and maintaining airflow into the nasal or oral cavity.

In various embodiments, a device of the invention also includes gas flow control valves such as check valves and a pressure transducer(s) to measure and/or control pressure inside the tank or chamber and a safety release valve to prevent over-pressurization of the chamber and subsequent damage and user injury.

In some embodiments of the invention, an APAP device comprises additional features integrated in such device worn by the user, including a clock, alarm, lights and or sound to awaken the patient, video camera to record user activity, speaker to play music, electronic display screen to play video, means to darken the lens to serve as eye guards for assisting the user in falling asleep and waking up.

Devices of the invention can also automatically set (titrate) the required nasal or oral pressure level to overcome the pharyngeal critical closing pressure thus eliminating pharyngeal obstruction. In embodiments, a device of the invention can also monitor, record and report device usage as well as other data, such as apnea or hypopnea events, oxygen saturation, sleep stage, etc. All data can be transmitted electronically via communication ports or wirelessly via computer or telephonically such as via a cell phone. Devices of the invention can also provide filtered clean air and/or moisture and heat to the pressurized breathable air. In further embodiments, devices of the invention can also be coupled with an oral appliance, such as for treatment of snoring or sleep apnea. An exemplary oral appliance may interact with oral tissue such as control of mandible position or tongue movement, or supply air to the oral cavity via the oral appliance. For example, the device may control tongue movement to mitigate tongue intrusion into the airway thus minimizing the gas pressure required to prevent obstruction of the pharynx.

It is an object of embodiments of the invention to provide a comfortable and aesthetically pleasing and inconspicuously designed eyewear to encourage use to increase user compliance, and eliminate air supply tube, power cord, and face mask encasing the users nose.

It is a further object of embodiments of the invention to provide a positive pressure device to be worn by the user to treat sleep apnea.

It is a further object of embodiments of the invention to provide a positive pressure device that is self-contained and battery powered where the entire system is contained in a single unit and inconspicuously designed as eyewear.

It is a further object of embodiments of the invention to provide a device that does not involve using a facemask that covers the nose that can be dislodged during sleep.

It is a further object of embodiments of the invention to provide a positive pressure device that does not require a large diameter air supply tube (hose) and allows the user to be unencumbered during sleep.

It is a further object of embodiments of the invention to provide a self-contained positive pressure device that utilizes pressurized air contained in an air chamber to provide positive air pressure to treat sleep apnea.

It is a further object of embodiments of the invention to provide an air chamber in the form of eyewear or headgear to improve device appearance.

It is a further object of embodiments of the invention to provide a self-contained positive pressure device that comprises multiple or dual lenses to form said air chamber.

It is a further object of embodiments of the invention to provide a self-contained positive pressure device that comprises means to prevent light transmission through said lens when needed.

It is a further object of embodiments of the invention to provide an alternative self-contained positive pressure device in the form of headgear that comprises pressurized air chamber and other components.

It is a further object of embodiments of the invention to provide a self-contained positive pressure device that comprises means to deliver pressurized air to the nasal and or oral cavity via tubes.

It is a further object of embodiments of the invention to provide a self-contained positive pressure device that comprises means to add moisture and/or heat to air delivered to the user.

It is a further object of embodiments of the invention to provide a self-contained positive pressure device that comprises sensors to detect breathing and apnea and hypopnea events.

It is a further object of embodiments of the invention to provide a self-contained positive pressure device that comprises a system including electronics, pumps and regulated valves to maintain and deliver pressurized air to eliminate airway occlusion.

It is a further object of embodiments of the invention to provide a self-contained positive pressure device that incorporates an auto titration feature to provide individualized appropriate air pressure to prevent occlusion.

It is a further object of embodiments of the invention to provide a self-contained positive pressure device that comprises means for information communication including wireless data transmission.

It is a further object of embodiments of the invention to provide a self-contained positive pressure device that comprises means to provide time and alarm features.

It is a further object of embodiments of the invention to provide a self-contained positive pressure device that comprises means to monitor EEG and oxygen saturation.

It is a further object of embodiments of the invention to provide self-contained system to connect to an oral appliance to control tongue occlusion thus minimizing positive pressure requirements.

### BRIEF DESCRIPTION OF THE DRAWINGS

FIG. 1 is a schematic block diagram of components of an Autonomous Positive Air Pressure System in one embodiment of the present invention.

FIG. 2 is a component operation table in one embodiment of the present invention.

FIG. 3A is a front perspective view of an Autonomous Positive Air Pressure System configured with any eyewear housing in one embodiment of the present invention.

FIG. 3B is a cross-sectional view along section A-A of FIG. 3A.

FIG. 4 is a combination perspective and schematic view of integrated components within an Autonomous Positive Air Pressure System configured with eyewear housing in one embodiment of the present invention.

FIG. 5 is a perspective view of an Autonomous Positive Air Pressure System configured and worn as eyewear by a user in one embodiment of the present invention.

### DETAILED DESCRIPTION OF THE INVENTION

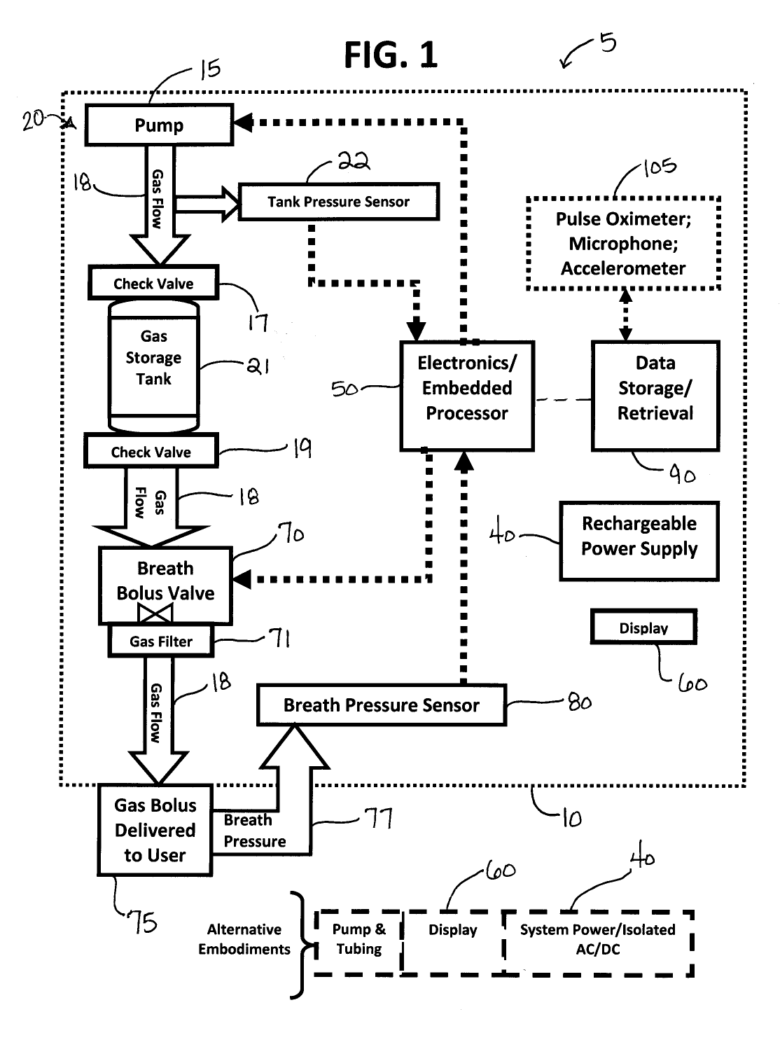

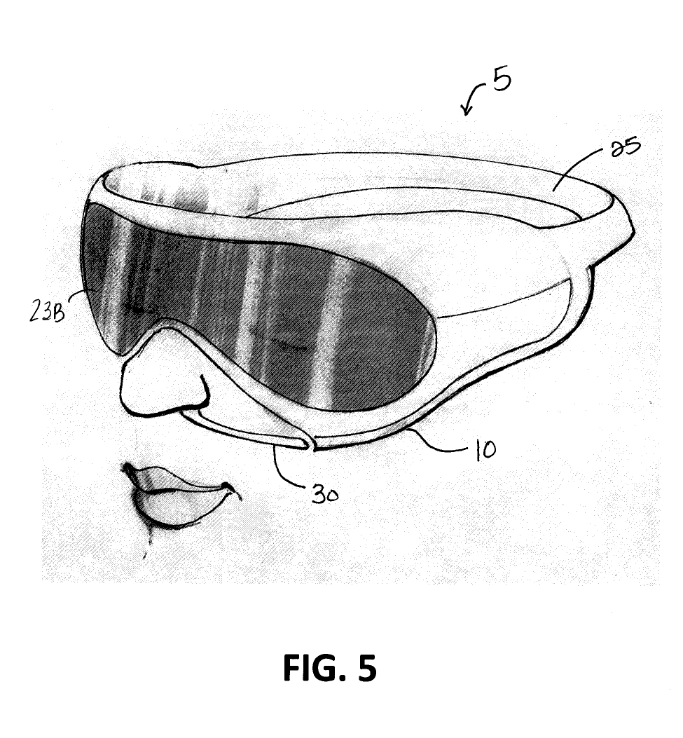

Referring to FIGS. 1, and 3-5, an Autonomous Positive Air Pressure device 5 in one embodiment of the invention inconspicuously pressurizes and delivers air to a user's nasal or oral cavity. In one embodiment, integrated components are concealed within a housing 10 of the device 5 configured as eyewear. In alternative embodiments, the device may be configured as headwear, arm wear, leg wear or worn on the torso. A flexible and/or adjustable attachment device 25 may be used to secure device 5 to the body.

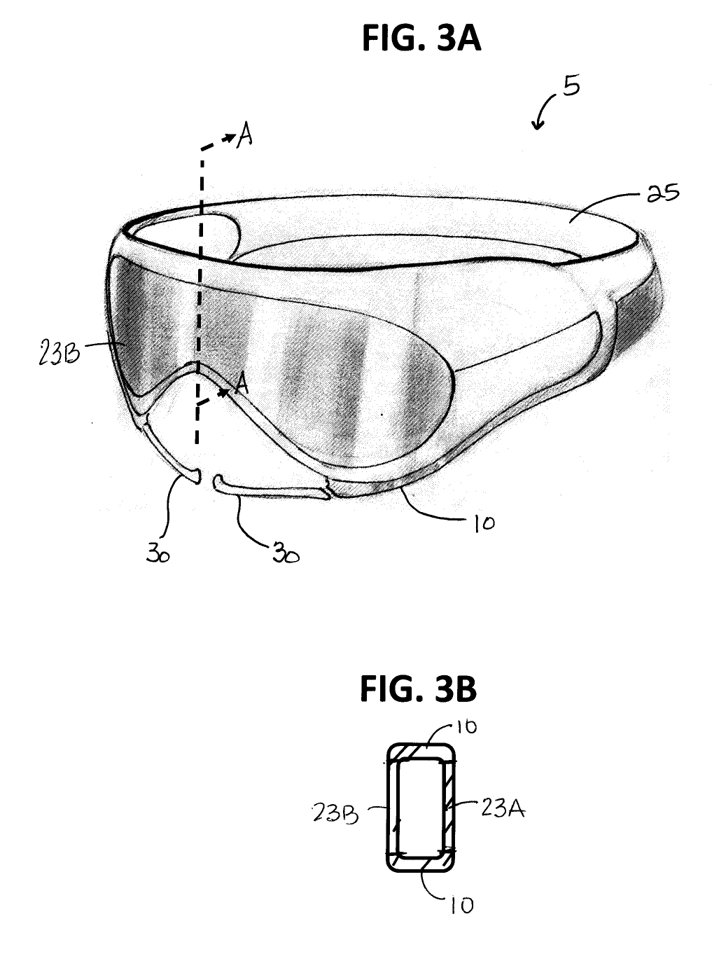

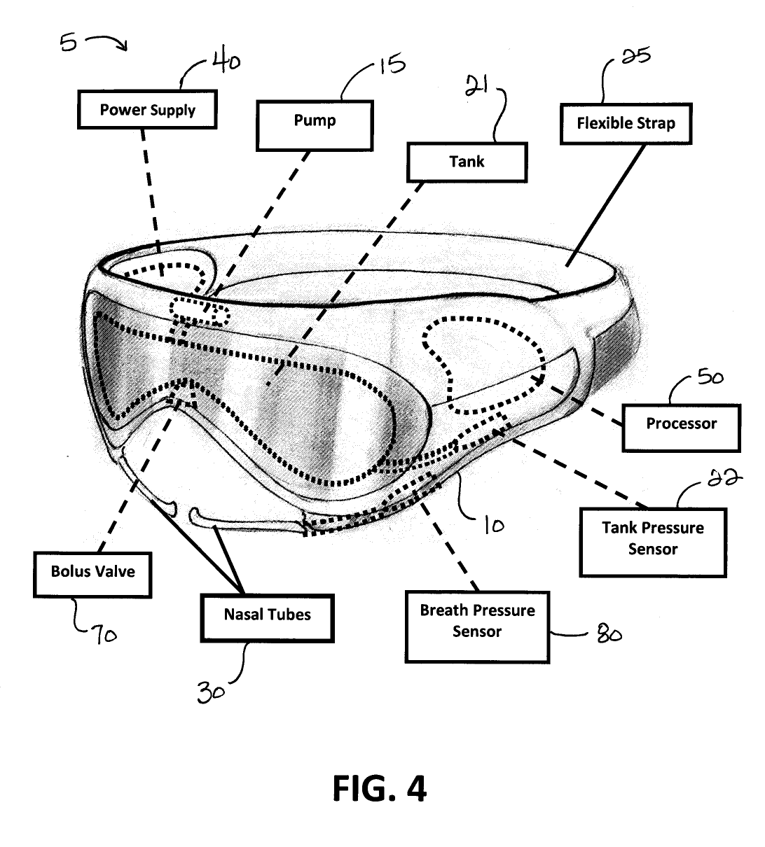

Device 5 includes a gas supply system 20 having a gas storage tank or chamber 21 integrated into housing 10. In embodiments the housing 10 may comprise Grilamid® material (EMS-GRIVORY) or cellulose acetate propionate (Eastman Chemical Company), or Ethylene Vinyl Acetate (EVA) foam or Thermoplastic elastomers (TPEs). The eyewear housing 10 may also comprise comfortable deformable padding material(s) to interface and conform to the users body contour that can evenly distribute pressure from the eyewear across the users body (e.g. face), provide softness to improve the feel of the device, and potentially absorb secretions (such as sweat) from the users body. Referring to FIGS. 3B and 4, in an exemplary eyewear embodiment, gas supply system 20 includes a chamber 21 that is formed, in part, by inner lens 23A and outer lens 23B, which are separated and sealed within a portion or portions of the housing 10, forming a hollow enclosure therebetween. In one embodiment the lenses are polycarbonate. Each lens in one embodiment is transparent but may also become opaque such as by electronically darkening the lens or by other means. In other embodiments, the lenses may be translucent or opaque.

In embodiments an APAP device 5 of the invention includes a pump 15 to maintain positive pressure in chamber 21. Gas flow 18 from pump 15 and through the gas supply system 20 and its components to a user is shown schematically in FIG. 1. An inlet check valve 17 and outlet check valve 19 may be provided for controlling the gas flow 18 at the entry and exit ports of chamber 21, respectively. Pump and valves of the invention may include, in various embodiments, components of Takasago Electric (Nagoya Japan), Smart Products, Inc. (Morgan Hill, Calif.) and/or Lee Products (Westbrook, Conn.). In alternative embodiments, a bellows-type component may also be used to compress air. In some exemplary embodiments, all of the components may be integrated within said housing 10. In some embodiments, one or more of the components may be located outside of the housing 10 as necessary or desired, such as, for example, a power source or pump source.

A breath bolus valve 70 may be coupled to the gas supply system 20 to control gas flow 18 as gas bolus 75 is delivered to a user. In some embodiments a gas filter 71, such as filter materials from Superior Felt & Filtration, LLC (Ingleside, Ill.) filters gas flow 18 from bolus valve 70, to remove undesired substances such as particles, bacteria and viruses.

Referring to FIGS. 3A, 4 and 5, tubes 30 are connected to the lower part of the housing 10 to direct gas flow 18 (such as pressurized air) of gas bolus 75 to a user's nasal or oral cavity. Tubes 30 are preferably flexible to allow device 5 movement during sleep. The shape of the tubes 30 can also be formed to assist in further opening nostrils. Appropriate materials are used to dampen vibration and pump or valve noises. Appropriate material can be used to cushion the device against the user's face.

In various embodiments, a microprocessor 50 (and related electronics) such as from Texas Instruments (Dallas, Tex.) provides centralized control of the integrated electronic components of the device 5, such as in response to one or more input signals. In some embodiments, a chamber pressure sensor 22, a breath pressure sensor 80 monitoring breath pressure 77, and/or data storage device 90 are coupled to microprocessor 50. In some embodiments, the microprocessor 50 is operably coupled with pump 15 and valve 70, for example, to control gas flow. The microprocessor 50 may be coupled with one or more additional components of the device 5, as necessary or desired.

Device 5 may be configured to include means for data storage, data conversion, data management, data display and/or data communication. For example, data storage device 90 may be coupled to a variety of optional data transceivers 105 including a pulse oximeter, microphone, accelerometer and computing devices, including mobile computing devices. Data connections may be wired or wireless. Data may also be represented in one or more display units 60 integrated with or externally connected to device 5.

In some embodiments microprocessor 50 may also receive pressure data from breath pressure sensor 80 and gas supply source pressure sensor 22. Such sensors are of the type as may be available from ServoFlow Corp. (Lexington, Mass.).

A power supply source 40 is preferably rechargeable and integrated in device 5. In some embodiments, exemplary power supply sources 40 include power sources such as those available from Micro Power Electronics (Beaverton, Oreg.).

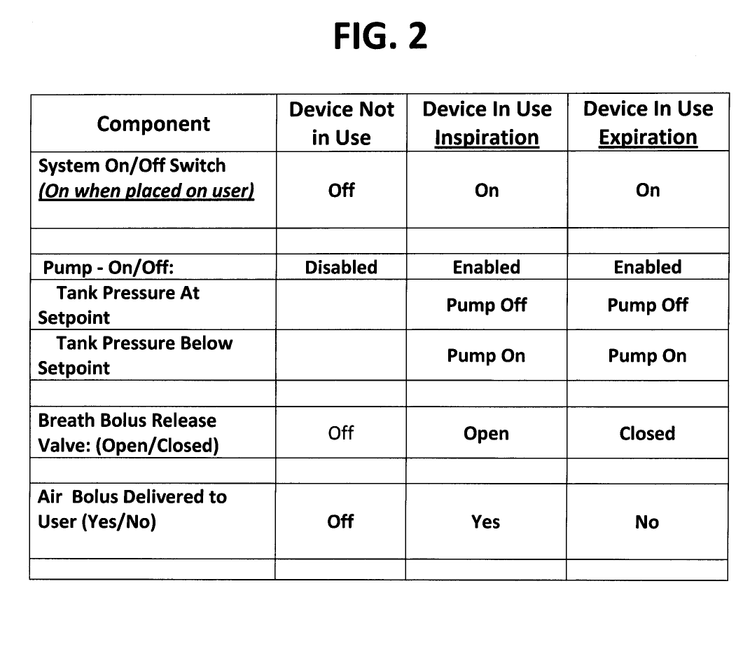

Referring to FIG. 2, a component operational control table illustrates exemplary component status during non-use, user inspiration and user expiration.

In still further embodiments of the invention, a number and variety of components may be further integrated into a wearable device 5 to provide user comfort or information functionality. Such exemplary additional components include sleep detection sensor, apnea sensor, hypopnea sensor, pulse oximeter monitor, clock, alarm, biological clock light, radio, video player, music player, safety valve to release excess gas pressure, check valves to control gas flow direction, air filters to clean air supplied to the user, electronic auto titration controller of gas supply source pressure, wireless connection to transmit data, patient use data recorder, nostril opener, gas moisturizer, air pressure adjuster, sleep stage monitor and data recorder, power source and device noise muffling material.

Accordingly, while the invention has been described with reference to the structures and processes disclosed, it is not confined to the details set forth, but is intended to cover such modifications or changes as may fall within the scope of the following claims.

What is claimed is:

1. A device comprising a housing configured as at least one of eyewear, headwear and arm wear and a pressurized gas supply system self-contained within the housing and operatively connected to at least one of a nasal air tube and oral cavity tube.

2. The device of claim 1, wherein the pressurized gas supply system comprises at least one of a chamber and tank.

3. The device of claim 2, wherein the pressurized gas supply system is configured and located in the position of one or more eyewear lenses.

4. The device of claim 1, wherein the pressurized gas supply system includes pressurized ambient air.

5. The device of claim 4, wherein the pressurized ambient air includes moisture.

6. The device of claim 4, wherein moisture includes at least one of water and pharmaceutical or medicant.

7. Device of claim 4, wherein the pressurized ambient air includes heat.

8. The device of claim 1, wherein the housing is configured as at least one of eyeglasses and goggles that include an eyewear lens space, side walls and frame.

9. The device of claim 8, wherein the housing configured as eyeglasses or goggles comprises coupling for external devices.

10. The device of claim 9, wherein the external devices includes at least oral appliances and gas channel.

11. The device of claim 10, wherein oral appliances includes at least one of snoring treatment and sleep apnea treatment devices.

12. The device of claim 4, wherein the pressurized gas supply system includes entry and exit ports for said gas.

13. The device of claim 12, wherein an exit port includes a valve.

14. The device of claim 13, wherein the valve is electronically controlled.

15. The device of claim 12, wherein the exit port includes a check valve controlling release of gas.

16. The device of claim 1, further comprising a pump to pressurize gas in the gas supply system, one or more pressure sensors coupled to the gas supply system and one or more valves coupled to the gas supply system, all of which integrated in said housing.

17. The device of claim 16, further comprising at least one of electronic control, data storage, data conversion, data management, data display, electronic communication ports, gas filters to remove undesired substances including particles, bacteria and viruses, gas pressure sensors, breathing pressure sensors, gas temperature sensors, a sleep detection sensor, apnea sensor, hypopnea sensor, pulse oximeter monitor, clock, alarm, biological clock light, radio, video player, music player, safety valve to release excess gas pressure, electronic auto titration controller of gas supply source pressure, wireless connection to transmit data, patient use data recorder, nostril opener, gas moisturizer, air pressure adjuster, sleep stage monitor and data recorder, power source and device noise muffling material.

18. The device of claim 1, further comprising an integrated power source and without physical tethers to external sources of power and gas.

19. The device of claim 1, further comprising non-integrated power source and non-integrated pump source.
